(19) United States
(12) Patent Application Publication
(10) Pub. No.: US 2005/0209609 A1
(43) Pub. Date: Sep. 22, 2005

(54) Foreign body retrieval devices

(75) Inventor: Michael J. Wallace, Bellaire, TX (US)

Correspondence Address: MARK T. GARRETT;FULBRIGHT AND JAWORSKI L.L.P, 600 CONGRESS AVENUE, SUITE 2400, AUSTIN, TX, 78701, US

(73) Assignee: Board of Regents, The University of Texas System

(21) Appl. No.: 11/064,062

(22) Filed: Feb. 23, 2005

Related U.S. Application Data
(60) Provisional application No. 60/547,215, filed on Feb. 24, 2004.

(51) Int. Cl. A61B 17/24

(52) U.S. Cl. 606/113

(57) ABSTRACT

Foreign body retrieval devices suited for initially engaging a foreign body at any location along the length of the foreign body. In one embodiment, a foreign body retrieval device that includes a retriever having a hook at one end; and a snare having an element that is configured to substantially surround, and that substantially surrounds, a portion of the hook when the snare and the retriever are used together to capture a foreign body. In another embodiment, a foreign body retrieval device that includes a retriever having a closed-arm hook at one end; and a snare having an element that is configured to engage the closed-arm hook. Other embodiments are included.

### CROSS-REFERENCE(S) TO RELATED APPLICATION(S)

This application claims priority to U.S. Provisional Patent Application Ser. No. 60/547,215, filed Feb. 24, 2004, the entire contents of which are expressly incorporated by reference.

### BACKGROUND OF THE INVENTION

1. Field of the Invention

The invention relates generally to the field of medical devices. More particularly, it relates to foreign body retrieval devices that are capable of initially engaging a foreign body at a location other than the end(s) of the foreign body.

2. Description of Related Art

During medical procedures that utilize catheters, guidewires, pacemaker leads, or other medical devices, a portion of the device can sometimes break off and be left within the patient. The detached portion may then travel within the patient's vascular system and come to rest in a luminal organ, vein or artery, and usually at a branching point or in the heart. Leaving these foreign bodies within the patient can be quite harmful, and may result in complications like sepsis, perforation, thrombosis, arrhythmias, myocardial necrosis, or even death. Therefore, it is necessary and urgent to remove the foreign body from the patient.

The Amplatz GOOSE NECK Snare, commercially available from ev3 Inc. (4600 Nathan Lane North Plymouth, Minn. 55442-2920) is one such foreign body retrieval device. See U.S. Pat. No. 5,171,233 to Amplatz et al. (1992), which is incorporated by reference. A shortcoming of this snare is that it can initially engage a foreign body only at the end or ends of the foreign body. This can make the initial engagement difficult. The same is true of the snare disclosed in U.S. Pat. No. 5,342,371 to Welter et al. (1994), which is incorporated by reference.

The snare in U.S. Pat. No. 5,562,678 to Booker (1996), which is incorporated by reference, is designed to allow for the initial engagement of a foreign body at a point along the foreign body other than its end or ends.

### SUMMARY OF THE INVENTION

In some embodiments, the present foreign body retrieval devices comprise, consist of, or consist essentially of a retriever having a hook at one end; and a snare having an element that is configured to substantially surround, and that substantially surrounds, a portion of the hook when the snare and the retriever are used together to capture a foreign body.

In other embodiments, the present foreign body retrieval devices comprise, consist of, or consist essentially of a retriever having a closed-arm hook at one end; and a snare having an element that is configured to engage the closed-arm hook.

In still other embodiments, the present foreign body retrieval devices comprise, consist of, or consist essentially of a catheter having a passageway system; a retriever slidably positionable in the passageway system, the retriever having a hook at one end; and a snare slidably positionable in the passageway system, the snare having an element that is configured to substantially surround, and that substantially surrounds, a portion of the hook when both the element and the hook are positioned outside of the passageway system.

In some embodiments, the present foreign body retrieval devices comprise, consist of, or consist essentially of a catheter having a passageway system; a retriever slidably positionable in the passageway system, the retriever having a closed-arm hook at one end; and a snare slidably positionable in the passageway system, the snare having an element that is configured to engage the closed-arm hook.

Additional embodiments of the present foreign body retrieval devices, and details associated with those embodiments, are set forth below.

### BRIEF DESCRIPTION OF THE DRAWINGS

The following drawings illustrate by way of example and not limitation. The use of identical reference numerals does not necessarily indicate an identical structure. Rather, the same reference numeral may be used to indicate a similar feature or a feature with similar functionality.

FIG. 1A is a front perspective view showing one of the present foreign body retrieval devices. The catheter is depicted partially in cross section.

FIG. 1B shows an example of a segment of material that is not open.

FIG. 2 is a partial front perspective view of another of the present foreign body retrieval devices.

FIG. 3 is a partial front perspective view of still another of the present foreign body retrieval devices.

FIG. 4 is a top view of one of the present elements of one of the present snares.

FIG. 5 is a partial front view of one of the present hooks of one of the present retrievers.

FIG. 6 is a partial front perspective view of one of the present foreign body retrieval devices that includes a retriever having a bent portion.

FIG. 7 is a partial front view of another version of the bent portion of one of the present retrievers.

FIG. 8 is a cross-sectional top view of a catheter suited for use with certain of the present foreign body retrieval devices.

FIG. 9 is a cross-sectional top view of another catheter suited for use with certain of the present foreign body retrieval devices.

FIG. 10 is a cross-sectional top view of versions of the present retrievers and snares that are configured to mate with each other such that they can move together and operate in a pre-determined orientation.

FIG. 11 is a cross-sectional top view of the versions of the present retrievers and snares shown in FIG. 10 positioned in one version of a catheter suited for use with certain of the present foreign body retrieval devices.

FIG. 12A is a partial front view of a catheter suited for use with certain of the present foreign body retrieval devices, the catheter having a side-arm adapter coupled to its distal end.

FIG. 12B depicts a dilator that may be used consistently with the present foreign body retrieval devices.

FIGS. 13-16 show stages of one manner of how one version of the present foreign body retrieval devices may be operated to capture a foreign body.

FIGS. 17-20 show stages of another manner of how one version of the present foreign body retrieval devices may be operated to capture a foreign body.

### DESCRIPTION OF ILLUSTRATIVE EMBODIMENTS

The terms “comprise” (and any form of comprise, such as “comprises” and “comprising”), “have” (and any form of have, such as “has” and “having”), and “include” (and any form of include, such as “includes” and “including”) are open-ended linking verbs. As a result, a device that “comprises,” “has,” or “includes” one or more elements possesses those one or more elements, but is not limited to possessing only those one or more elements. Likewise, an element of a device that “comprises,” “has,” or “includes” one or more features possesses those one or more features, but is not limited to possessing only those one or more features.

Thus, and by way of example, a foreign body retrieval device “comprising” a retriever having a hook at one end; and a snare having an element that is configured to substantially surround, and that substantially surrounds, a portion of the hook when the snare and the retriever are used together to capture a foreign body has, but is not limited to having only, the recited features. That is, the foreign body retrieval device possesses at least the recited features, but does not exclude other features that are not expressly recited.

The terms “a” and “an” are defined as one or more than one unless this disclosure explicitly requires otherwise.

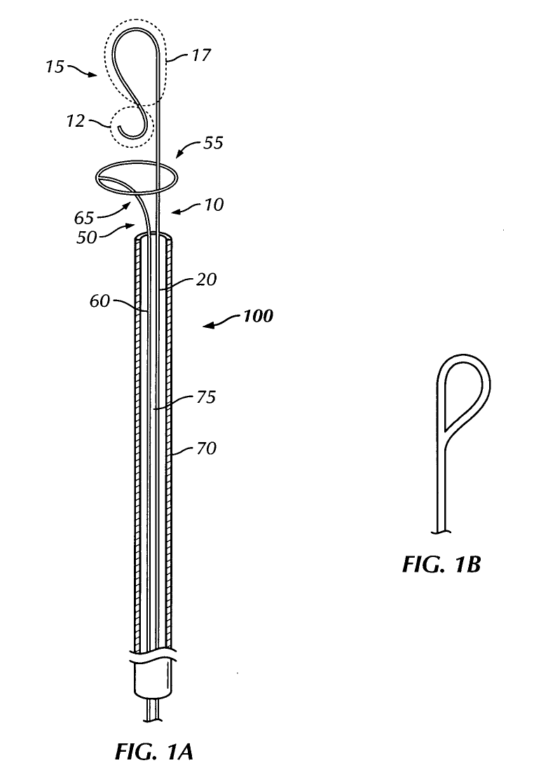

Certain of the present foreign body retrieval devices include at least a retriever having a hook at one end; and a snare that has an element that is configured to substantially surround, and that substantially surrounds, a portion of the hook when the snare and the retriever are used together to capture a foreign body. The term “substantially” is defined as at least close to (and can include; preferably within 10% of, more preferably within 1% of, and most preferably within 0.1% of) a given value or state. FIG. 1A shows one example of such a foreign body retrieval device.

Foreign body retrieval device 100 includes retriever 10 and snare 50. Retriever 10 has a hook 15 at one of its ends. Snare 50 has an element 55 that is configured to substantially surround, and that substantially surrounds, a portion of hook 15 when snare 50 and retriever 10 are used together to capture a foreign body. An explanation of how retriever 10 and snare 50 can be used together to capture a foreign body is described below.

A “hook” is defined as a segment of material that is open (meaning that the hook is capable of initially engaging a foreign body at a location between the ends of the foreign body; see FIG. 1B for an example of a segment of material that is not open) and that is bent back, at least to some extent, on itself. There is no hook, according to this definition, disclosed in the figures, “Summary of the Invention” or “Detailed Description” sections of U.S. patent application Publication No. US 2002/0188262 A1, which is incorporated by reference. None of the elements of the “retrieval catheter” disclosed in those sections includes a segment of material that is open and that is bent back, at least to some extent, on itself.

Furthermore, while element 30 of reversible needle's eye snare 10 in U.S. Pat. No. 5,562,678 qualifies as a hook as defined above, threader 45 does not qualify as one of the present elements because threader 45 is not configured to substantially surround, and does not substantially surround, a portion of element 30 when the two are used to capture a foreign body, or when the two are positioned outside of sheath 60. Instead, threader 45 is configured to be threaded through—and in use is threaded through—element 30 when the two are used to capture a foreign body, or when the two are positioned outside of sheath 60.

In the embodiment shown in FIG. 1A, element 55 is a loop. A loop is defined as a structure that is at least somewhat curved and at least substantially closed (it can be completely closed). If the loop has a break—such that it is not completely closed—it is preferable that the break be positioned near where the loop meets the snare shaft. The loop may have any suitable shape, including but not limited to an oval or a circle.

Continuing with the embodiment of foreign body retrieval device 100 shown in FIG. 1A, hook 15 includes a main hook portion 17 and a reverse hook tip portion 12. Retriever 10 has a retriever shaft 20 that, in this embodiment, is substantially straight. Retriever shaft 20 is threadably positionable in element 55. In the embodiment shown in FIG. 1A, retriever shaft 20 is actually threaded through (and thus is also threadable through) the loop that is element 55.

Hook 15 also is one embodiment of one of the present closed-arm hooks. A closed-arm hook is defined as one of the present hooks that is made from either a single strand or filament (such as a wire), or if a single strand is bent and doubled over, or if two or more strands are used, any space between adjacent strands or adjacent segments of the same strand is sufficiently small that the element (e.g., element 55) of the snare with which the hook is designed to operate cannot be threaded through the space. In broad embodiments, this is true where the element cannot be threaded through the space when the element is in its unconstrained state. In other embodiments, this is true where the element cannot be threaded through the space in a constrained state, such as a completely constrained state. Closed loop 30 of reversible needle's eye snare 10 in U.S. Pat. No. 5,562,678 does not qualify as one of the present closed-arm hooks because the space between the adjacent segments of the material that forms closed loop 30 is too large—one of the present elements 55 can be threaded through the space. One of the present snares that is used with one of the present retrievers that has one of the present closed-arm hooks at one end of the retriever may be characterized as having an element that is configured to engage that closed-arm hook.

Snare 50 includes a snare shaft 60 that, in this embodiment, is substantially straight along the majority of its length, but also includes a bent portion 65 that links the straight portion of the snare shaft to element 55. In this embodiment, bent portion 65 is oriented in such a way that the substantially straight portion of snare shaft 60 is centered (or at least substantially centered) with respect to the loop that is element 55. By configuring snare 50 with a snare shaft that has such a bent portion, it is easier to maintain the orientation of element 55 with respect to hook 15 than if element 55 is connected to a substantially straight shaft with no bent portion, as shown in FIG. 3. This follows because if snare shaft 60 in FIG. 1A is rotated about its axis, that rotation will not cause element 55 to move substantially; this is in contrast to what would happen if snare shaft 60 of the version of snare 50 shown in FIG. 3 were rotated in the same fashion.

As FIG. 1A shows, snare 100 may also include a catheter 70 having a passageway system 75 in which both retriever 10 and snare 50 are slidably positionable (and, in this embodiment, positioned). Passageway system 75 has a single lumen in this embodiment.

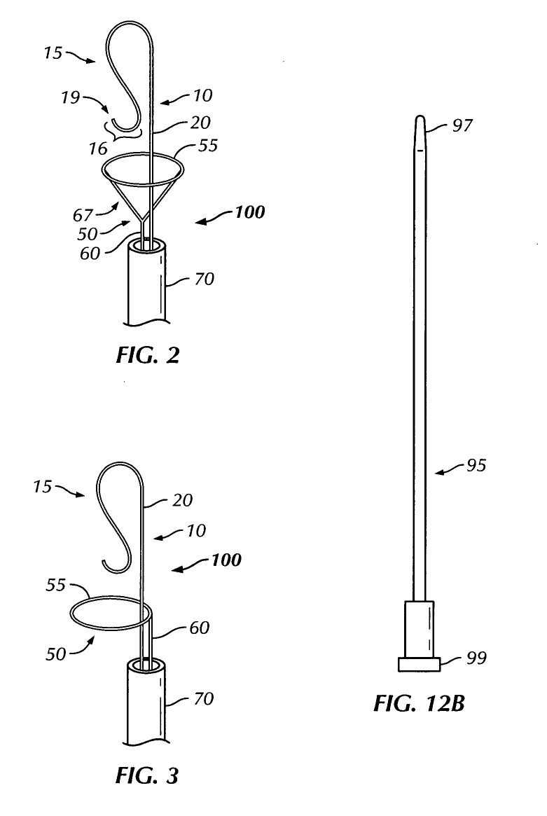

Other suitable versions of foreign body retrieval device 100 are shown in FIGS. 2 and 3. These figures show alternative embodiments of snare 50, while the embodiments of retriever 10 remain the same as those shown in FIG. 1A. Snare 50 in FIG. 2 includes snare shaft 60, which has a substantially straight portion and, more specifically, is substantially straight. This version of snare 50 also includes a V-shaped portion 67 that links snare shaft 60 to element 55. V-shaped portion 67 includes 2 strands (such as wires) that are connected to snare shaft 60 and to oppositely-positioned points on element 55. FIG. 2 shows that hook 15 includes a termination point 19 and a flared portion 16 that extends progressively farther from retriever shaft 20 moving toward termination point 19. In this embodiment, reverse hook tip portion 12 (not labeled in FIG. 2) includes flared portion 16. V-shaped portion 67 of snare 50 serves the same function as bent portion 65 of the version of snare 50 shown in FIG. 1A: it helps to maintain the orientation of element 55 with respect to hook 15.

FIG. 3 shows a version of foreign body retrieval device 100 that includes a version of snare 50 having a substantially straight snare shaft 60 that is directly connected to element 55. As shown in this figure, element 55 may meet snare shaft 60 at an angle greater than zero degrees. More specifically, element 55 may meet snare shaft 60 at an angle greater than 30 degrees, 45 degrees, or 60 degrees. Even more specifically, element 55 may meet snare shaft 60 at an angle of approximately 90 degrees as shown in FIG. 3. The term “approximately” is defined as at least close to (and can include; preferably within 10% of, more preferably within 1% of, and most preferably within 0.1% of) a given value or state. An Amplatz GOOSE NECK Snare may be used for the version of snare 50 depicted in FIG. 3. As this figure shows, retrieval shaft 20 is threaded (and thus is threadably positionable) through element 55.

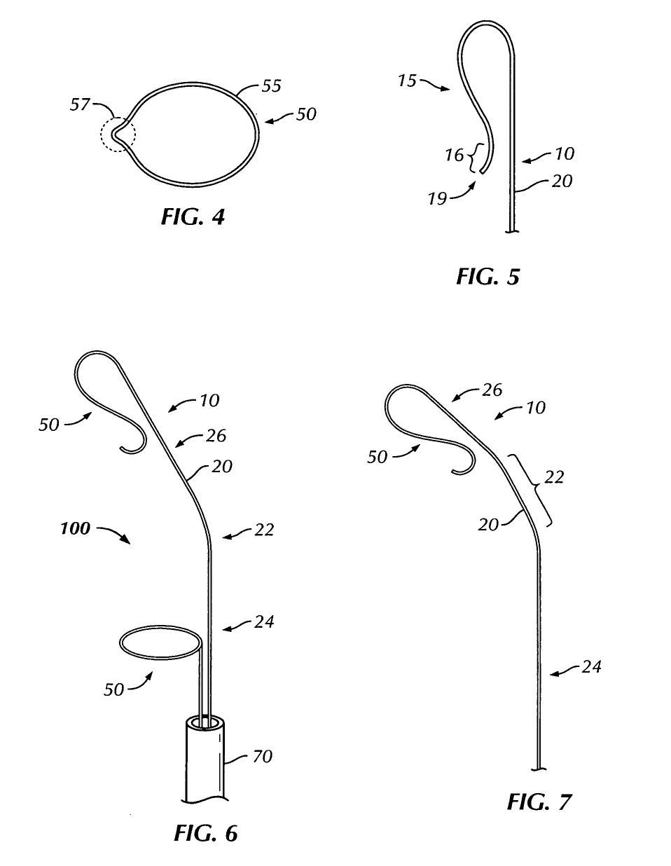

Element 55 of snare 50 may have any suitable shape, as discussed above. FIG. 4 shows a top view of a version of snare 50 in which element 55 has a somewhat oval shape. In addition, this figure shows that element 55 may be provided with one or more kinks 57. Kink 57 functions to provide a controlled “collapse” point for element 55 as it is drawn into a catheter in order to secure a captured foreign body (discussed in more detail below). Such kinks are provided in the Amplatz GOOSE NECK Snares, and are well known to those of ordinary skill in the art. A kink of the type illustrated in FIG. 4 may be placed near each location where V-shaped portion 67 meets element 55 in the version of snare 50 shown in FIG. 2.

FIG. 5 shows another version of hook 15 of retriever 10 that may be used consistently with the present foreign body retrieval devices. The embodiment of hook 15 shown in this figure lacks reverse hook tip portion 12 shown in FIGS. 1A-3. This version of hook 15 does, however, include termination point 19 and flared portion 16, which extends progressively farther from retriever shaft 20 moving toward termination point 19.

The versions of the present retrievers shown in FIGS. 1A-3 are suited for use when the body cavity or space of interest is relatively small, such as a blood vessel (vein or artery), bile duct, or the ureter. When one of the present foreign body retrieval devices is to be used in a cavity or space that is large, such as the urinary bladder, it will be beneficial to be able to reach areas of such a space that are spaced laterally from the hook without having to tilt or reposition the foreign body retrieval device. The version of retriever 10 shown in FIG. 6 helps to accomplish this goal.

Retriever 10 shown in FIG. 6 has a retriever shaft 20 that includes a substantially straight portion 24, which may also be described as a substantially straight proximal portion. Retriever shaft 20 also includes a bent portion 22 that links substantially straight portion 24 to hook 50. That link can be indirect, as shown in FIG. 6, because a substantially straight distal portion 26 may be positioned between hook 50 and bent portion 22. Furthermore, bent portion 22 may have a series of bends, as shown in FIG. 7. As a result, the bends in the series can be exposed as needed. For example, the first bend in a series (i.e., the bend that is closest to hook 50) can be exposed from a catheter in which retriever 10 is slidably positioned without necessarily exposing additional bends. Then, as needed, additional bends along shaft 20 may be exposed afterwards. As a result, a single retriever (having appropriately-spaced bends) can be used in many different situations. In this same regard, if the space-of-interest is relatively small, the retriever can be operated without exposing any of the bends.

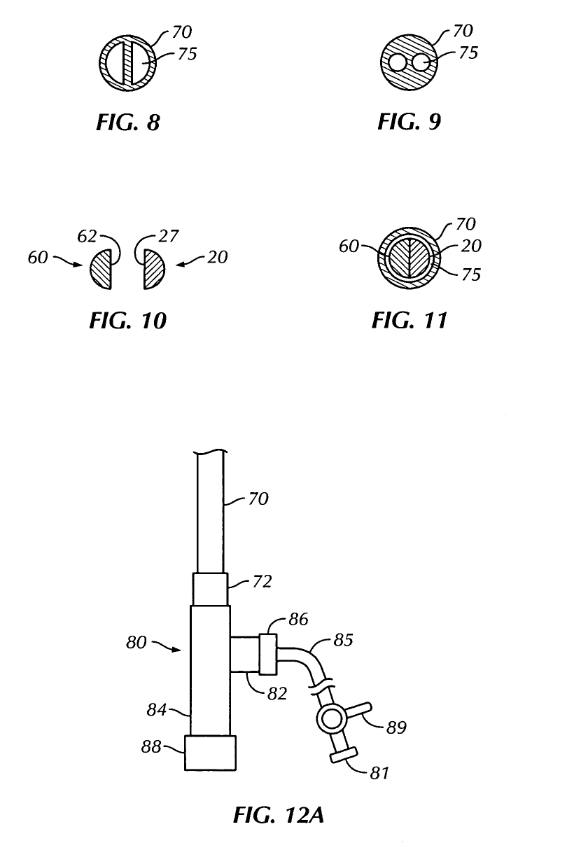

Any catheter that is used with the present foreign body retrieval devices can be provided with a passageway system that includes one or more lumens. FIG. 1A shows a version of passageway system 75 of catheter 70 that includes a single lumen. FIGS. 8 and 9 show versions of passageway system 75 that include multiple lumens—specifically two. The two lumens of the version of passageway system 75 shown in FIG. 8 have opposing “D” shapes. The two lumens of the version of passageway system 75 shown in FIG. 9 have side-by-side circular shapes. Any other suitable lumen shapes may be utilized.

The snares and retrievers of the present foreign body retrieval devices may be configured in any fashion that is suitable for allowing them to operate effectively together. One example of how to achieve such a configuration is shown in FIG. 10. FIG. 10 depicts cross-sectional views of snare shaft 60 of snare 50 and retriever shaft 20 of retriever 10. Snare shaft 60 is provided with a snare shaft surface 62, and retriever shaft 20 is provided with a retriever shaft surface 27. Both surfaces are configured to mate with each other such that the snare and retriever can move together and operate in a pre-determined orientation. In this embodiment, that configuration is achieved by making both surfaces flat.

FIG. 11 shows how the versions of snare 50 and retriever 10 shown in FIG. 10 may “mate” with each other within passageway system 75 of catheter 70. Using this embodiment, the size of element 55 may be reduced as much as possible because when retriever shaft 20 is rotated such that hook of retriever 10 swivels and faces a new direction, snare shaft 60 will be forced to move as well, thus maintaining the pre-determined orientation of the element with respect to the hook. Maintaining a pre-determined orientation in this fashion is not necessarily possible using the passageway system of FIG. 9 and a retriever and a snare that each have round shafts. Other configurations of the present snares and retrievers may be implemented to achieve the same mating function.

The catheters that may be used with the present foreign body retrieval devices, and that in certain embodiments are part of the present foreign body retrieval devices, may be adapted to be coupled to traditional side-arm adapters, such as a Tuohy-Borst side-arm adapter, commercially available from Cook. FIG. 12 shows such a version of catheter 70. The distal end of catheter 70 is provided with a hub 72, which may be built into the catheter or attached to the catheter as a separate piece. Hub 72 may be provided with a male or a female Luer lock, or any other structure suitably configured to allow for the coupling of a fitting that posses, for example, a hemostasis valve and/or a side-arm adapter, all of which are well known in the art. (See U.S. Pat. No. 5,098,393 (incorporated by reference), which discloses a traditional hemostasis valve and side-arm tube having a stopcock. See U.S. Pat. No. 5,324,262 (incorporated by reference) for the same purpose. See also U.S. Pat. No. 5,391,152 (incorporated by reference) for an example of another hemostasis valve and side-arm adapter. Proximal fitting 80 may be coupled to catheter 70 through hub 72, and may include a side-arm port 82 and a main port 84. Both ports may be configured (e.g., appropriately-sized) such that a retriever, a snare, or both are slidably insertable through them and into catheter 70. Side-arm port 82 may also be used for the injection of contrast during a procedure (for diagnostic purposes), saline solution, medicinal solution, or any other suitable solution. The ends of the side-arm and main ports may be provided with hubs 86 and 88, respectively, each of which may be adapted to be coupled to a valve, such as a hemostasis valve. For example, a hemostasis valve (not visible) may be provided within either or both of hubs 86 and 88. A tube (e.g., a flexible tube) 85 may be coupled to side-arm port 82 (and, more specifically, to hub 86), and a stopcock 89 may be coupled to tube 85. A hub 81 may be coupled to an end of stopcock 89 for attachment of other medical devices (e.g., a syringe).

The snares and retrievers of the present foreign body retrieval devices may be made of any suitable material, or combination of materials, including the nickel-titanium alloy sold under the name NITINOL. Other non-ferrous metals (i.e., magnetic resonance imaging (MRI) compatible) may also be used. When the present retrievers and snares are made of a metal or metal alloy, they may be described as being made of wire. When a thermal memory material such as the nickel-titanium alloy NITINOL is used for either instrument, those of ordinary skill in the art will understand how to heat treat the material to impart desired thermal-memory properties to it. The treatment may be tailored to ensure that some of the material retains its superelastic property. Where portions of material are joined together, welding or soldering may be used to effect the connection(s).

The present retrievers and snares, and the catheters that may be used with—and that in certain embodiments are part of—the present foreign body retrieval devices may be sized to fit a given application. By way of example only, a catheter having an outer diameter of 5- to 7-French (F) and an inner diameter of 3- to 5-F (e.g., a single lumen catheter) may be used with a retriever having a retriever shaft of 0.018-0.022 inches and a snare having a snare shaft of 0.018-0.022 inches. Both hook 15 and element 55 may have diameters of 10 millimeters.

The present foreign body retrieval devices may comprise kits that include one of the present retrievers, one of the present snares, and one or more of the following: one of the present catheters (having some or all of the features discussed above, such as a fitting having one or more hemostasis valves, one or more side-arm ports, and one or more tubes with a stopcock), a dilator configured to work with the catheter, an introducer sheath, a dilator configured to work with the introducer sheath, and an enclosure (e.g., a bag made of a polymeric material) for holding these items. For example, one version of the present foreign body retrieval devices that comprises a kit includes one of the present retrievers; one of the present snares; one of the present catheters that includes a fitting having a proximal hemostasis valve and a side-arm to which a tube having a stopcock and stopcock hub have been coupled; a dilator configured to work with the catheter; and an enclosure in which these items are held. One or more, up to all, of the items in a given kit may be sterilized.

An examples of a suitable introducer sheath that can be used consistently with the present foreign body retrieval devices in certain applications is the PINNACLE® introducer sheath commercially available from SCIMED®/Boston Scientific Corporation. If an introducer sheath is used during insertion of one of the present foreign body retrieval devices, a dilator may be used in combination with the sheath. U.S. Pat. Nos. 5,098,392 and 5,391,152 disclose examples of sheaths and dilators that may be used together. Catheters with tapered distal ends (the end farthest from the operator) that are also angled for directional control may be used as the present catheters. A generic representation of a dilator 95 that may be used in combination with one of the present catheters is depicted in FIG. 12B. Dilator 95 is provided with a tapered or conically-shaped distal end 97. The proximal end of dilator 95 is provided with a hub 99 that may be configured to engage hub 72 of catheter 70 shown in FIG. 12A. The conical or tapered shape of distal end 97 may protrude beyond the distal end of catheter 70 in use, and may help to gradually expand the puncture site for insertion of catheter 70. In certain applications, it may be possible to dispense with the use of a sheath and dilator, and use only one of the present catheters and a dilator for introducing one of the present retrievers and one of the present snares to a target location.

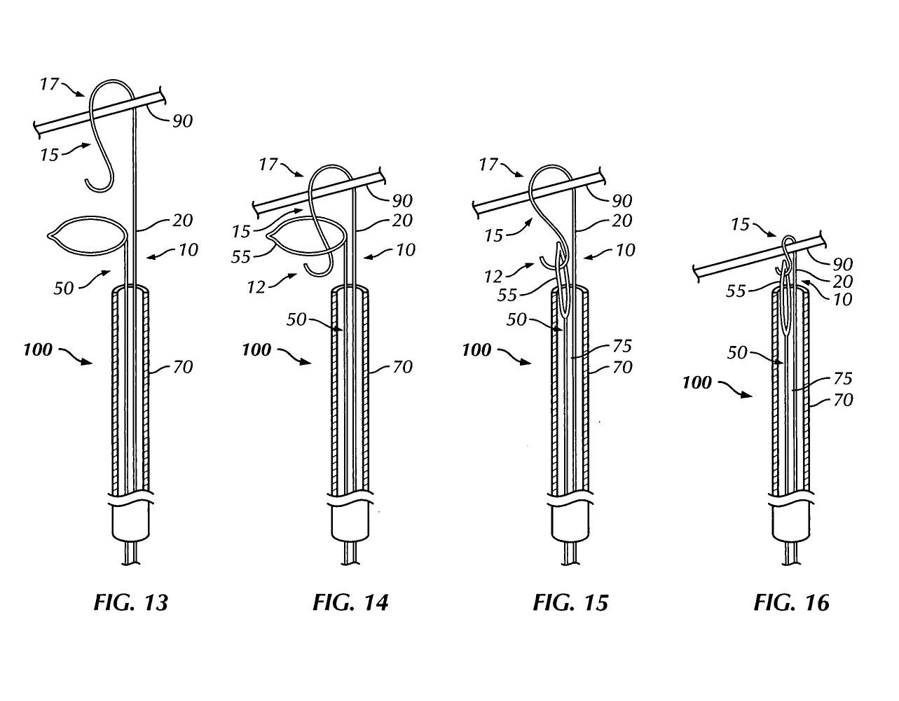

An example of how one version of the present foreign body retrieval devices may be operated to capture a foreign body is shown in stages in FIGS. 13-16. First, a delivery catheter, such as catheter 70 shown in the figures, may be advanced as far as possible to the target site. This may occur after first using a sheath and dilator, or a dilator with catheter 70. A steerable guidewire also may be used in order to gain access to the target site, as is well-known to those of ordinary skill in the art.

Once the target site is reached, any guidewire that has been used may be replaced with one of the present retrievers, one of the present snares, or both, in order to maintain the access that is achieved. As shown in FIG. 13, retriever 10 of foreign body retrieval device 100 may be advanced such that it engages foreign body 90 (anywhere along the length of foreign body 90) within main hook portion 17. Next, with the position of the retriever maintained, snare 50 and catheter 70 may be advanced forward until element 55 of the snare (e.g., the loop shown in FIG. 14) substantially (and, in this case, completely) surrounds at least a portion of hook 15. In FIG. 14, the portion of hook 15 that is surrounded using element 55 is the portion that is closest to retriever shaft 20.

Next, as shown in FIG. 15, while maintaining the position of catheter 70 and retriever 10, snare 50 may be retracted into passageway system 75 of catheter 70. As this retraction takes place, element 55 will be collapsed and withdrawn into passageway 75. Next, as shown in FIG. 16, while maintaining the position of snare 50 and catheter 70, retriever 10 may be retracted, thereby reducing the size of the main portion of hook 15, and drawing foreign body 90 closer to the distal tip of catheter 70. The operator of the foreign body retrieval device may advance and retract catheter 70, snare 50, and retriever 10 as desired until captured foreign body 90 is sufficiently secure. Foreign body retrieval device 100 and foreign body 90 may then be retracted from the target site and removed, using an access sheath to facilitate that process as desired.

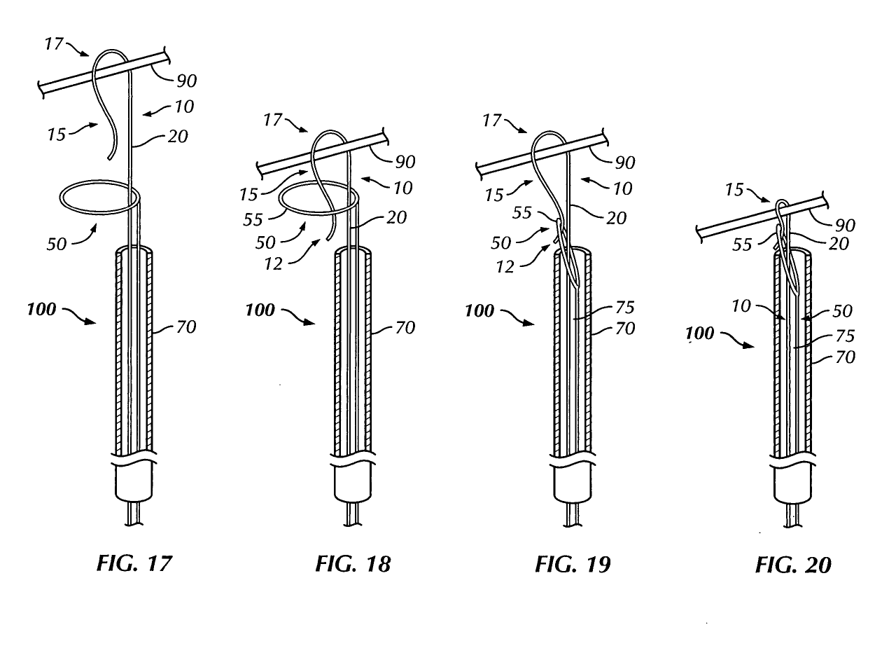

As an alternative way to operate one of the present foreign body retrieval devices to capture a foreign body is shown in stages in FIGS. 17-20. This embodiment of foreign body retrieval device 100 differs from the version depicted in FIGS. 13-16 in two respects: the configuration of element 55 and the configuration of hook 15. Hook 15 in FIGS. 17-20 is the same as the hook depicted in FIG. 5. The configuration of element 55 in FIGS. 17-20 is the same configuration that is depicted in FIG. 3. Specifically, retrieval shaft 20 is threaded through element 55.

Advancement to the target site and the initial engagement of foreign body may take place as described above. This initial engagement is shown in FIG. 17. Next, with the position of the retriever maintained, snare 50 and the delivery catheter (e.g., catheter 70) may be advanced forward until element 55 of the snare (e.g., the loop shown in FIG. 18) substantially (and, in this case, completely) surrounds at least a portion of hook 15. In FIG. 18, the portion of hook 15 that is surrounded using element 55 is the portion that is closest to retriever shaft 20. Furthermore, element 55 also surrounds a portion of retriever shaft 20 because retriever shaft 20 is threaded through element 55.

Next, as shown in FIG. 19, while maintaining the position of catheter 70 and retriever 10, snare 50 may be retracted into passageway system 75 of catheter 70. As this retraction takes place, element 55 will be collapsed and withdrawn into passageway 75. This retraction also narrows the gap between (a) the portion of hook 15 that was initially closest to retriever shaft 20 and (b) retriever shaft 20. Next, as shown in FIG. 20, while maintaining the position of snare 50 and catheter 70, retriever 10 may be retracted, thereby reducing the size of the main portion of hook 15, and drawing foreign body 90 closer to the distal tip of catheter 70. The operator of the foreign body retrieval device may advance and retract catheter 70, snare 50, and retriever 10 as desired until captured foreign body 90 is sufficiently secure. Foreign body retrieval device 100 and foreign body 90 may then be retracted from the target site and removed, using an access sheath to facilitate that process as desired.

It should be understood that the present foreign body retrieval devices are not intended to be limited to the particular forms disclosed. Rather, they are to cover all modifications, equivalents, and alternatives falling within the scope of the claims. For example, although two versions of the present hooks have been illustrated, other hook designs that fit the definition provided above may be used. Similarly, the present retrievers and snares may be provided with radiopaque markers—such as platinum, gold or tungsten coils surrounding a portion of the material from which the retrievers and/or snares are made—that make them easier to locate under fluoroscopic guidance. Furthermore, while the versions of element 55 shown in the figures are fixed in size (their shapes are deformable), those of ordinary skill in the art will understand that versions of element 55 that have variable sizes may also be used with any of the present snares.

The claims are not to be interpreted as including means-plus- or step-plus-function limitations, unless such a limitation is explicitly recited in a given claim using the phrase(s) “means for” or “step for,” respectively.

1. A foreign body retrieval device comprising:
a retriever having a hook at one end; and
a snare having an element that is configured to substantially surround, and that substantially surrounds, a portion of the hook when the snare and the retriever are used together to capture a foreign body.

2. The foreign body retrieval device of claim 1, where the retriever has a retriever shaft and a retriever shaft surface, the snare has a snare shaft and a snare shaft surface, and the retriever shaft surface is configured to mate with the snare shaft surface.

3. The foreign body retrieval device of claim 1, where the snare has a snare shaft, and the snare shaft has a proximal end and a bend located between the hook and the proximal end.

4. The foreign body retrieval device of claim 1, where the hook has a main hook portion and a reverse hook tip portion.

5. The foreign body retrieval device of claim 1, where the retriever has a retriever shaft that is substantially straight.

6. The foreign body retrieval device of claim 1, where the retriever has a retriever shaft with a substantially straight portion.

7. The foreign body retrieval device of claim 6, where the retriever shaft includes a bent portion that links the hook to the substantially straight portion.

8. The foreign body retrieval device of claim 1, where the snare includes a snare shaft and a V-shaped portion that links the element to the snare shaft.

9. The foreign body retrieval device of claim 1, where the element is a loop.

10. The foreign body retrieval device of claim 1, where the retriever has a retriever shaft, and the hook includes a flared portion that extends progressively farther from the retriever shaft moving toward the one end.

11. The foreign body retrieval device of claim 1, where the retriever is made of a nickel-titanium alloy.

12. The foreign body retrieval device of claim 1, where the snare is made of a nickel-titanium alloy.

13. A foreign body retrieval device comprising:
a retriever having a closed-arm hook at one end; and
a snare having an element that is configured to engage the closed-arm hook.

14. The foreign body retrieval device of claim 13, where the retriever has a retriever shaft and a retriever shaft surface, the snare has a snare shaft and a snare shaft surface, and the retriever shaft surface is configured to mate with the snare shaft surface.

15. The foreign body retrieval device of claim 13, where the snare has a snare shaft, and the snare shaft has a proximal end and a bend located between the hook and the proximal end.

16. The foreign body retrieval device of claim 13, where the closed-arm hook has a main hook portion and a reverse hook tip portion.

17. The foreign body retrieval device of claim 13, where the retriever has a retriever shaft with a substantially straight portion.

18. The foreign body retrieval device of claim 17, where the retriever shaft includes a bent portion that links the closed-arm hook to the substantially straight portion.

19. A foreign body retrieval device comprising:
a catheter having a passageway system;
a retriever slidably positionable in the passageway system, the retriever having a hook at one end; and
a snare slidably positionable in the passageway system, the snare having an element that is configured to substantially surround, and that substantially surrounds, a portion of the hook when both the element and the hook are positioned outside of the passageway system.

20. A foreign body retrieval device comprising:
a catheter having a passageway system;
a retriever slidably positionable in the passageway system, the retriever having a closed-arm hook at one end; and
a snare slidably positionable in the passageway system, the snare having an element that is configured to engage the closed-arm hook.
